(19) United States
(12) Patent Application Publication
(10) Pub. No.: US 2021/0282714 A1
(43) Pub. Date: Sep. 16, 2021

(54) BIOLOGICAL INFORMATION PRESENTATION DEVICE, BIOLOGICAL INFORMATION PRESENTATION METHOD, AND NON-TRANSITORY COMPUTER-READABLE MEDIUM

(71) Applicants: Hirohito Miyazaki, Aichi (JP); Takashi Aragai, Aichi (JP)

(72) Inventors: Hirohito Miyazaki, Aichi (JP); Takashi Aragai, Aichi (JP)

(73) Assignee: NIDEC MOBILITY CORPORATION, Aichi (JP)

(21) Appl. No.: 17/197,664

(22) Filed: Mar. 10, 2021

(30) Foreign Application Priority Data
Mar. 16, 2020 (JP) 2020-045594

(51) Int. Cl. A61B 5/00; G07C 5/08; B60K 35/00

(52) U.S. Cl. CPC A61B 5/6893; G07C 5/0825; B60K 35/00; A61B 2503/06; B60K 2370/178; A61B 5/7405; A61B 5/742; B60K 2370/167

(57) ABSTRACT

A biological information presentation device includes a biosensor configured to detect biological information of an occupant existing in a vehicle, and a second controller configured to present, via a presenter, a fact that biological information has been detected if the biosensor detects biological information.

### CROSS-REFERENCE TO RELATED APPLICATION

This application is based on Japanese Patent Application No. 2020-45594 filed with the Japan Patent Office on Mar. 16, 2020, the entire contents of which are incorporated herein by reference.

### FIELD

The present invention relates to a biological information presentation device, a biological information presentation method, and a biological information presentation program.

### BACKGROUND

There has ever been a major problem with a type of incident of leaving a child in a vehicle interior in which a parent gets out of the car while unintentionally leaving a child inside the car, and the parent forgets a child present in the vehicle interior.

To solve this problem, various countermeasures and researches are being carried out. For example, a technology has been disclosed in which a biosensor configured to detect biological information in a vehicle is provided, and if the biological information has been detected when an occupant turns off the engine of the vehicle or locks the door, it is determined that someone is left, and a warning to that fact is issued to the occupant.

However, with the technique disclosed in Japanese Patent Application Laid-Open No. 2000-185609, detection of leaving someone behind is started after the occupant expresses his/her intention to get out of the vehicle, and hence it takes time until warning the occupant about leaving someone behind. For this reason, there is a case of failing to warn the occupant against leaving someone behind before the occupant gets out of the vehicle and leaves the vehicle. When detection whether someone is left behind is performed after the engine is turned off, the determination time is too short to provide the accuracy enough to discriminate between a peripheral object and a living body, and there is a possibility of issuing a false warning or no warning.

### SUMMARY

One aspect of the present invention has been made in view of the above-mentioned problem, and an object of the present invention is to provide a technique capable of reliably warning the occupant against leaving someone behind during a period from when the occupant gets out of the vehicle to when the occupant leaves the vehicle.

The biological information presentation device according to the present invention includes a biosensor configured to detect biological information of an occupant existing in a vehicle, and a controller configured to present, via a presenter, the fact that biological information has been detected if the biosensor detects the biological information.

The biological information presentation method according to the present invention includes a step of detecting, by a biosensor, biological information of an occupant existing in a vehicle, and a control step of presenting, via a presenter, the fact that biological information has been detected if the biosensor detects the biological information.

According to one aspect of the present invention, it is possible to reliably warn the occupant against leaving someone behind during a period from when the occupant gets out of the vehicle to when the occupant leaves the vehicle.

### BRIEF DESCRIPTION OF THE DRAWINGS

FIG. 1 is a configuration diagram of a vehicle equipped with a biological information presentation device according to an embodiment of the present invention;

FIG. 2 is a view showing an example of a presenter of a vehicle equipped with a biological information presentation device according to an embodiment of the present invention;

FIG. 3 is a view showing an example of a change in a presentation mode in a presenter of a vehicle equipped with a biological information presentation device according to an embodiment of the present invention;

FIG. 4 is a view showing another example of a change in a presentation mode in a presenter of a vehicle equipped with a biological information presentation device according to an embodiment of the present invention;

FIG. 5 is a view showing yet another example of a change in a presentation mode in a presenter of a vehicle equipped with a biological information presentation device according to an embodiment of the present invention; and

FIG. 6 is a flowchart showing an example of processing of a biological information presentation device according to an embodiment of the present invention.

### DETAILED DESCRIPTION

Embodiments of the disclosure will be described with reference to the drawings. In the drawings, the identical or equivalent component is designated by the identical numeral. In embodiments of the disclosure, numerous specific details are set forth in order to provide a more thorough understanding of the invention. However, it will be apparent to one of ordinary skill in the art that the invention may be practiced without these specific details. In other instances, well-known features have not been described in detail to avoid obscuring the invention.

An embodiment of the present invention will be described below in detail with reference to the drawings. For convenience of explanation, members having the identical functions to those previously described are given the identical reference numerals, and the description thereof is sometimes omitted.

[Vehicle Configuration]

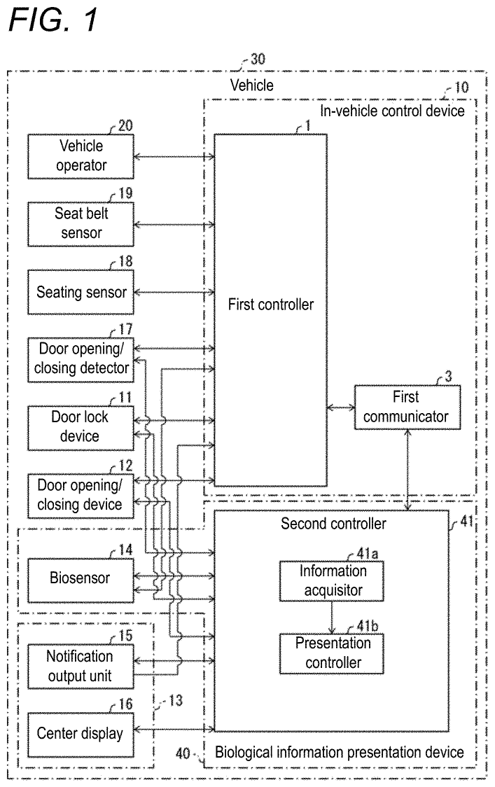

First, the configuration of a vehicle 30 equipped with a biological information presentation device 40 according to the embodiment will be described with reference to FIG. 1. FIG. 1 is a block diagram showing the configuration of the vehicle 30.

The vehicle 30 is, for example, an auto four-wheel vehicle. A living body (not illustrated) may exist in a vehicle interior of the vehicle 30. Examples of the living body include an infant, a pet, and an elderly person. Examples of the occupant include a parent of an infant, a pet owner, and a caregiver of an elderly person.

As shown in FIG. 1, the vehicle 30 is equipped with an in-vehicle control device 10, the biological information presentation device 40, a door lock device 11, a door opening/closing device 12, a notification output unit 15, a center display (also called an instrument panel, corresponds to the “display” of the claims 16, a door opening/closing detector 17, a seating sensor 18, a seat belt sensor 19, and a vehicle operator 20. However, the description of components unrelated to the present invention will be omitted.

[In-vehicle Control Device]

The in-vehicle control device 10 comprehensively controls the operation of each part of the vehicle 30.

As shown in FIG. 1, the in-vehicle control device 10 includes a first controller 1 and a first communicator 3. The first controller 1 includes a CPU and a memory or the like. The first controller 1 is connected to the door lock device 11, the door opening/closing device 12, the notification output unit 15, the center display 16, the door opening/closing detector 17, the seating sensor 18, the seat belt sensor 19, and the vehicle operator 20, and acquires state information regarding the state of each unit from each unit of the vehicle and operation information regarding the operation of each unit of the vehicle from the vehicle operator 20. The first controller 1 transmits control information to each unit of the vehicle, thereby controlling the operation of each unit of the vehicle 30.

The in-vehicle control device 10 is connected with the biological information presentation device 40 via the first communicator 3, and communicates with the biological information presentation device 40.

[Each Unit of Vehicle and Vehicle Operator]

The door lock device 11, the door opening/closing device 12, the door opening/closing detector 17, the seating sensor 18, the seat belt sensor 19, and the vehicle operator 20 will be described below. A biosensor 14, the notification output unit 15, and the center display 16 will be described later in another section.

The vehicle 30 is provided with a plurality of doors. Of them, the door of the driver seat, the door of the passenger seat, the door of the right back seat, the door of the left back seat of the vehicle 30, and the back door of the rear surface of the vehicle 30 can be locked/unlocked by the door lock device 11 of FIG. 1.

The doors of left and right back seats are power sliding doors, which can be opened and closed by the door opening/closing device 12 of FIG. 1. Hereinafter, the power sliding door is referred to as a “PSD”. The PSD of the right and left back seats may be a hinged door. In this case, the vehicle 30 does not need the door opening/closing device 12.

The door lock device 11 includes a mechanism for locking/unlocking the door of the vehicle 30 and a drive circuit for the mechanism. The door opening/closing device 12 includes a mechanism for causing the PSD of the vehicle 30 to open/close and a drive circuit for the mechanism.

A door switch not illustrated in FIG. 1 includes a door switch installed in each door. As an example, the door switch installed in the door is turned on when the door of the driver seat is in an open state, and the door switch installed in the door is turned off when the door is in a closed state. However, this does not limit the embodiment, and the door switch may be turned on when the door is in a closed state, and may be turned off when the door is in an open state. The same applies to other door switches included in the door switch.

The door opening/closing detector 17 detects the opening/closing operation of the door by detecting the state of the door switch described above. In other words, the door opening/closing detector 17 detects the opening/closing operation of each door by detecting the states of the plurality of door switches included in the door switch. For example, the door opening/closing detector 17 detects that an opening operation of the door of the driver seat has been made when the state of the door of the driver seat changes from the closed state to the open state, and detects that a closing operation of the door of the driver seat has been made when the state of the door of the driver seat changes from the open state to the closed state. The same applies to the other doors.

The seating sensor 18 detects that the occupant is seated in or has left the driver seat of the vehicle 30. The seating sensor 18 may be, for example, a pressure sensor incorporated in the seat or backrest of the driver seat of the vehicle 30.

The seat belt sensor 19 detects that the seat belt provided in the driver seat of the vehicle 30 is attached/detached.

(Vehicle Operator)

The vehicle operator 20 refers to each unit of the vehicle operated by the occupant of the vehicle 30. For example, the vehicle operator 20 includes an ignition (IG) switch, a power switch (main power supply (if the vehicle 30 is an EV vehicle) switch, auxiliary power switch, and the like), and a shift lever, and the like.

Operation information of each unit of the vehicle operated by the occupant is transmitted to the in-vehicle control device 10 and the biological information presentation device 40. Upon receiving the operation information, the first controller 1 transmits control information to each unit of the vehicle.

For example, when the shift lever is operated from the parking position to the drive position, information indicating that the shift lever has been operated is output to the biological information presentation device 40. When the IG switch is turned on or off, information indicating that the IG switch has been turned on or off is output to the biological information presentation device 40.

[Biological Information Presentation Device 40]

The biological information presentation device 40 includes the biosensor 14, and a second controller 41 configured to present, via a presenter 13 (notification output unit 15 and center display 16), which will be described later, the fact that the biological information has been detected when the biosensor 14 detects biological information.

(Biosensor)

The biosensor 14 is a sensor configured to detect biological information of the occupant existing in the vehicle interior. The biosensor 14 is, for example, a radio wave sensor, and may be incorporated in the backrest of the seat. The radio wave sensor irradiates a living body with radio waves and receives reflected waves from the living body, thereby detecting biological information. Examples of biological information include heart rate, blood pressure, respiratory rate, body temperature, pulse rate, and brain waves. Alternatively, the biosensor 14 may be a video sensor such as a camera provided on the dashboard or ceiling of the vehicle 30. In this case, biological information such as blood pressure may be detected from the video of the face of the living body captured by the camera or the like.

(Second Controller 41)

As shown in FIG. 1, the second controller 41 of the biological information presentation device 40 further includes an information acquisitor 41a and a presentation controller 41b. The door lock device 11, the door opening/closing device 12, the notification output unit 15, the center display 16, the door opening/closing detector 17, the seating sensor 18, the seat belt sensor 19, and the vehicle operator 20 are each connected also to the biological information presentation device 40.

The second controller 41 includes a CPU, a memory, or the like. The second controller 41 is connected with the first controller 1 and transmits/receives various types of information to/from the in-vehicle control device 10.

The information acquisitor 41a acquires status information of each unit of the vehicle from each unit of the vehicle. For example, the information acquisitor 41a acquires operation information indicating that each door has been operated from the door opening/closing detector 17. The information acquisitor 41a acquires information indicating that the occupant is seated in the seat or has left the seat from the seating sensor 18. As described above, the information acquisitor 41a acquires, from the seat belt sensor 19, information indicating that the seat belt has been attached/detached. The information acquisitor 41a acquires, from the vehicle operator 20, information indicating that each operator has been operated. The information acquisitor 41a acquires information indicating that, for example, the IG switch has been turned on or off or information indicating that the shift lever has been operated.

The presentation controller 41b refers to the state information of each unit of the vehicle received by the information acquisitor 41a, the operation information acquired from each operator of the vehicle, and the detection result of the biological information acquired from the biosensor 14, and determines whether or not to present to the presenter 13 and determines the presentation content. That is, when the biosensor 14 detects biological information, the presentation controller 41b of the biological information presentation device 40 presents, via the presenter 13, the fact that the biological information has been detected. It should be noted that the presentation described above will also be referred below to as presentation of “reminder”.

For example, the second controller 41 starts presentation via the presenter 13 when the information acquisitor 41a has acquired information indicating the occupant's intention to get on and the biosensor 14 has detected biological information. The information indicating the occupant's intention to get on may include information indicating at least any of activation of the travel power supply of the vehicle 30, activation of the auxiliary power supply of the vehicle, activation of the travel drive source of the vehicle, seating of the occupant, opening/closing of the door of the vehicle, unlock of the door of the vehicle, fastening of the seat belt of the vehicle, and operation of the operator of the vehicle.

For example, unlock of the door includes unlock of the door with a smart key, unlock of the door with a keyless entry system, and unlock of the door with a mechanical key. When the door is unlocked by the mechanical key, whether or not to start detection of the biological information by the biological sensor 14 may be settable by the occupant.

If the biological information in the vehicle is not detected by the biological sensor 14 even after a predetermined time has elapsed, the detection processing of the biological information may be canceled. Alternatively, the detection processing of the biological information may be canceled also when the door is relocked.

When the IG switch or the auxiliary power switch is turned on, it may be determined that the occupant's intention to get on has been indicated.

When the seating sensor 18 or the seat belt sensor 19 is turned on, it may be determined that the occupant's intention to get on has been indicated.

In addition, the detection processing of biological information by the biological sensor 14 may be started if information indicating opening/closing of the door, shift of the shift lever from the parking position to the drive position, and the like is acquired.

As described above, according to the biological information presentation device 40 of the embodiment, from when the occupant indicates the intention to get on, the biosensor 14 starts detection of biological information in the vehicle. Therefore, the biosensor 14 can start detection of biological information at an appropriate timing.

When the biosensor 14 detects biological information in the vehicle, the detection result is transmitted to the information acquisitor 41a of the second controller 41, and the presentation controller 41b starts the presentation of reminder in the presenter 13. The mode of presentation will be described later.

The second controller 41 may continue presentation via the presenter 13 at all times while the second controller 41 is acquiring information indicating the occupant's intention to get on and the biosensor 14 is detecting biological information. In this case, the occupant can always recognize the presence of the living body in the vehicle 30 while existing in the vehicle 30, it is possible to prevent someone from being left behind. Alternatively, the second controller 41 may repeat the presentation via the presenter 13 intermittently while the second controller 41 is acquiring the information indicating the occupant's intention to get on and the biosensor 14 is detecting biological information. In this case, the occupant can repeatedly recognize that the living body is present in the vehicle 30 while the occupant exists in the vehicle 30, and hence the living body is prevented from being left behind, and the power consumption by the biosensor 14 can be suppressed low.

The presentation content presented via the presenter 13 may further include the biological information of the occupant. For example, the biological information such as the pulse, heart sound, and blood pressure of the living body having been detected may be displayed on the center display 16. In this case, if there is an abnormality in the biological information, it is possible to respond at an early stage. Alternatively, whether the living body is awake or asleep may be displayed on the center display 16. Whether the living body is awake or asleep can be distinguished by the depth of respiration. For example, reference data obtained by measuring the swelling of the chest caused by the respiration of each living body during waking and sleeping are stored, and when the swelling caused by the respiration of the chest is equal to or more than a threshold value, the living body may be determined to be asleep. In this case, the occupant can grasp the state of the living body in addition to the presence of the living body.

Furthermore, during the presentation operation via the presenter 13, if the second controller 41 acquires information indicating the occupant's intention to get out, the second controller 41 may change the mode of presentation via the presenter 13. The information indicating the occupant's intention to get out may include information indicating at least any of stopping of the travel power supply of the vehicle 30, stopping of the auxiliary power supply of the vehicle 30, stopping of the travel drive source of the vehicle 30, leaving the seat of the occupant, operation of the door lock of the vehicle 30, opening/closing of the door of the vehicle 30, removal of the seat belt of the vehicle 30, and shift of the shift lever of the vehicle 30 to the parking position. It should be noted that the changed presentation described above will also be referred below to as presentation of “warning”.

For example, if the door is locked in a state where the IG switch or the auxiliary power switch is turned off while reminder is being presented, it may be determined that the occupant has indicated his/her intention to get out. Alternatively, if the shift lever is shifted to the parking position and the door is closed in a state where the IG switch or the auxiliary power switch is turned off while reminder is being presented, it may be determined that the occupant has indicated his/her intention to get out. When an authentication key is taken out of the vehicle with the IG switch remaining in the on state while reminder is being presented, it may be determined that the occupant has indicated his/her intention to get out.

As described above, the presentation via the presenter 13 may be shifted from the presentation of reminder to the presentation of warning. This makes it possible to appropriately warn the occupant when an incident of leaving someone behind is highly likely to occur.

The change of mode of presentation via the presenter 13 may include any of change in color of display on the center display 16 that displays the presentation content presented via the presenter 13, change in size of display on the center display 16, change in design of display on the center display 16, and change in output of warning sound from the notification output unit 15.

For example, while the fact that the biosensor 14 detects biological information in the vehicle 30 is displayed on the center display 16, if the information acquisitor 41a acquires information indicating that the IG switch has been turned off or if the information acquisitor 41a acquires information indicating that the door has been unlocked, the living body is highly likely to be left behind. Therefore, the display mode of the center display 16 may be changed to a display mode more attention-getting to the occupant.

Alternatively, the notification output unit 15 may set off a warning sound. An example of display mode change in the center display 16 will be described later with reference to FIGS. 3 to 5.

The second controller 41 may stop the presentation via the presenter 13 if the biosensor 14 becomes in a state of not detecting biological information, or if the presentation setting is canceled by an operation of the occupant. For example, when the living body (infant or the like) existing in the vehicle 30 gets off, the presentation via the presenter 13 is stopped. In addition, the occupant may operate a presentation cancel switch or the like provided in the vehicle 30 to stop presentation at the presenter 13. However, in this case, only the presentation of reminder is canceled, and the detection of biological information by the biosensor 14 may remain activated.

As described above, the presentation of reminder is cancelled also when the presentation of reminder is shifted to the presentation of warning.

[Presenter]

As described above, the presenter 13 includes the center display 16 and the notification output unit 15.

(Notification Output Unit)

The notification output unit 15 is a device configured to output sound such as a buzzer and a horn or voice. The notification output unit 15 may be a device configured to voice-output the content of the reminder or warning to the occupant against leaving someone behind.

(Center Display)

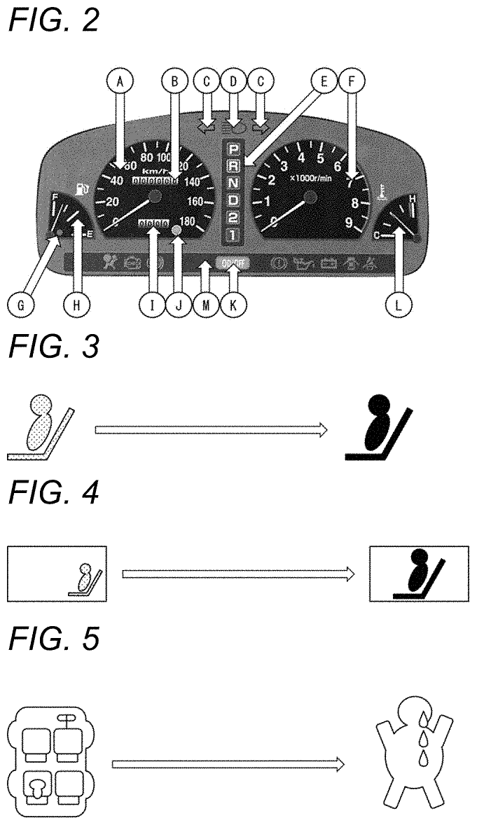

FIG. 2 shows an example of the center display 16 mounted on the vehicle 30. The display mode in the center display 16 will be described below with reference to FIG. 2. However, FIG. 2 is an example of display of the center display 16, and display of the center display 16 varies depending on the manufacturer and model of the vehicle.

The center display 16 is mounted in a cockpit in front of the driver seat of the vehicle 30. As shown in FIG. 2, the center display 16 includes display areas A to M, and displays various information regarding the vehicle 30. For example, travel speed of the vehicle 30 is shown in the area A. The area B is an odometer and indicates the total travel distance of the vehicle 30. The direction indicated by the turn signal is displayed in the area C. Display as to whether or not the headlight is in high beam is displayed in the area D. The position of the shift lever is displayed in the area E. The engine speed is displayed in the area F. The warning about the remaining amount of fuel is displayed in the area G. The remaining amount of fuel is displayed in the area H. The section travel distance is displayed in the area I, which is a trip meter. A reset button of the trip meter is displayed in the area J. The overdrive on/off is displayed in the area K. The temperature of the engine cooling water is displayed in the area L. Furthermore, when biological information is detected by the biosensor 14, a mark (also called a reminder mark) indicating that fact is displayed in the area M. As described above, if information indicating the occupant's intention to get out is acquired while the reminder mark described above is being displayed, the reminder mark is changed to a warning mark for warning against leaving someone behind.

FIGS. 3 to 5 show examples of change in the display mode of the biological information in the area M.

For example, when the biosensor 14 detects biological information in the vehicle, as shown in the left of FIG. 3, a reminder mark indicating that a living body is being seated on the seat may be displayed in a light color such as yellow. If the information acquisitor 41a acquires information indicating the occupant's intention to get out while the reminder mark is being displayed, as shown in the right of FIG. 4, the reminder mark may be changed to a warning mark with the same design as the reminder mark on the left of FIG. 4 in a dark color such as red, which gets more attention to the occupant.

If the biosensor 14 detects biological information in the vehicle, as shown on the left of FIG. 4, a small reminder mark indicating that the living body is being seated on the seat may be displayed. Then, if the information acquisitor 41a acquires information indicating the occupant's intention to get out while the reminder mark on the left in FIG. 4 is being displayed, as shown in the right of FIG. 4, the mark on the left of FIG. 4 may be changed to a large-sized warning mark of the same design.

If the biosensor 14 detects biological information in the vehicle, as shown in the left of FIG. 5, a reminder mark indicating that a living body is being seated on one of the four seats in the vehicle and indicating the position of the seat on which the living body is being seated may be displayed. Then, if the information acquisitor 41a acquires information indicating the occupant's intention to get out while the reminder mark on the left of FIG. 5 is being displayed, as shown in the right of FIG. 5, the reminder mark may be changed to a warning mark having a design more attention-getting to the occupant that is different from that of the reminder mark on the left of FIG. 5. The warning mark of the design shown on the right of FIG. 5 is an example, and a warning mark changed to another design may be displayed.

Furthermore, if the information acquisitor 41a acquires information indicating the occupant's intention to get out while the reminder mark on the left of FIG. 3, the left of FIG. 4, or the left of FIG. 5 is displayed, in place of the display shown in the right of FIG. 3, the right side of FIG. 4, or the right side of FIG. 5 on the center display 16 or in addition to any of them, the notification output unit 15 may set off a warning sound for warning against leaving someone behind. The notification output unit 15 may be configured to voice-output the content of reminder or warning against leaving someone behind to the occupant. Alternatively, the notification output unit 15 may be configured to simply set off a warning sound.

As described above, the center display 16 may be provided with a cancel button for canceling the presentation in the presenter 13, and the presentation of reminder or warning against leaving someone behind may be canceled when the occupant operates the cancel button. Alternatively, the presentation of reminder may be cancelled by the operation of the occupant, but the presentation of warning may be configured to be non-cancelable.

As described above, according to the biological information presentation device 40 having the above-described configuration, the occupant can recognize the presence of a living body by presentation of reminder that the biosensor 14 has detected biological information in the vehicle 30 in a normal travel state. In the case where reminder is not displayed despite the presence of a living body in the vehicle, the occupant can find the failure of the biosensor 14. Furthermore, if the information acquisitor 41a of the second controller 41 acquires information indicating the occupant's intention to get out while reminder is being presented, it is possible to smoothly shift from the presentation of reminder to the presentation of warning against leaving someone behind. Therefore, before the occupant gets out of and leaves the vehicle 30, it is possible to warn the occupant about the fact that the living body exists in the vehicle 30 and the risk of leaving the someone behind.

[Biological information presentation method (reminder display method)]

Next, an example of biological information presentation processing (reminder presentation processing) in the biological information presentation device 40 will be described.

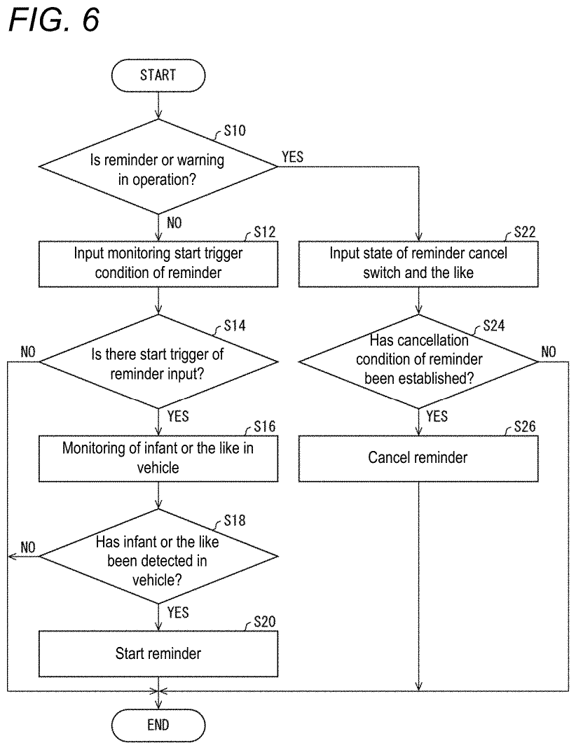

FIG. 6 is a flowchart illustrating the flow of processing in the biological information presentation device 40. The flow of the biological information presentation processing will be described below with reference to FIG. 6.

(Step S10)

When the vehicle 30 starts, the process proceeds to step S10.

In step S10, the second controller 41 determines whether or not presentation of reminder or warning in the presenter 13 is in operation. If presentation of reminder or warning in the presenter 13 is in operation (YES in step S10), the process proceeds to step S12. If presentation of reminder or warning in the presenter 13 is not in operation (NO in step S10), the process proceeds to step S22.

(Step S12)

In step S12, a trigger condition for starting the monitoring of reminder is input. Subsequently, the process proceeds to step S14.

(Step S14)

In step S14, it is determined whether or not a trigger for starting reminder has been input. That is, it is determined whether or not information indicating the occupant's intention to get on has been acquired. If a trigger for starting reminder has been input (information indicating the occupant's intention to get on has been acquired, i.e., YES in step S14), the process proceeds to step S16. If a trigger for starting reminder has not been input (information indicating the occupant's intention to get on has not been acquired, i.e., NO in step S14), the process ends.

(Step S16)

In step S16, monitoring of the infant in the vehicle, i.e., detection of biological information in the vehicle 30 by the biosensor 14 is started. Subsequently, the process proceeds to step S18.

(Step S18)

In step S18, the second controller 41 determines whether or not the biosensor 14 has detected biological information in the vehicle 30. If the biosensor 14 has detected biological information in the vehicle 30 (YES in step S18), the process proceeds to step S20. If the biosensor 14 has not detected biological information in the vehicle 30 (NO in step S18), the process ends.

(Step S20)

In step S20, since the biosensor 14 has detected biological information in the vehicle 30, presentation of reminder to the presenter 13 starts. The presentation may be a display on the center display 16 as shown on the left of FIG. 3, the left of FIG. 4, or the left of FIG. 5, as described above.

(Step S22)

In step S22, a state of the operation on a reminder cancellation switch and the like is input. Subsequently, the process proceeds to step S24.

(Step S24)

In step S24, it is determined whether or not a reminder cancellation condition has been established. If the reminder cancellation condition has been established (YES in step S24), the process proceeds to step S26. If the reminder cancellation condition has not been established (NO in step S24), the process ends. The reminder cancellation condition will be described later.

(Step S26)

In step S26, the presentation of reminder is canceled.

Thus, the processing of the biological information presentation method ends.

According to the above-mentioned biological information presentation method, the occupant can recognize the presence of a living body by presentation of reminder that the biosensor 14 has detected biological information in the vehicle 30 in a normal travel state. In the case where reminder is not presented despite the presence of a living body in the vehicle, the occupant can find the failure of the biosensor 14. Furthermore, if the information acquisitor 41a of the second controller 41 acquires information indicating the occupant's intention to get out while reminder is being presented, it is possible to smoothly shift from the presentation of reminder to the presentation of warning against leaving someone behind. Therefore, before the occupant gets out of and leaves the vehicle 30, it is possible to warn the occupant about the fact that the living body exists in the vehicle 30 and the risk of leaving the someone behind.

[Implementation Example by Software]

The in-vehicle control device 10 and the controller (in particular, the first controller 1, the information acquisitor 41a, and the presentation controller 41b) of the biological information presentation device 40 may be implemented by a logic circuit (hardware) formed in an integrated circuit (IC chip) or the like, or may be implemented by software.

In the latter case, the in-vehicle control device 10 and the biological information presentation device 40 include a computer that executes a command of a program that is software implementing each function. This computer includes, for example, one or more processors and a computer-readable recording medium storing the program. In the computer, the processor reads and executes the program from the recording medium, thereby achieving the object of the present invention.

As the processor, for example, a central processing unit (CPU) can be used. As the recording medium described above, a “non-temporary tangible medium”, for example, a read only memory (ROM), a tape, a disk, a card, a semiconductor memory, a programmable logic circuit, or the like can be used.

A random access memory (RAM) that expands the program may be further provided. The program described above may be supplied to the computer via any transmission medium (communication network, broadcast wave, and the like) capable of transmitting the program. Note that an aspect of the present invention can also be implemented in the form of a data signal embedded in a carrier wave in which the program described above is embodied by electronic transmission.

[Summary]

[Aspect 1]

The biological information presentation device of the present invention includes a biosensor configured to detect biological information of an occupant existing in a vehicle, and a controller configured to present, via a presenter, the fact that biological information has been detected if the biosensor detects the biological information.

According to the configuration described above, when a living body (infant) exists in a vehicle, for example, in a back seat, the occupant can always recognize the presence of the living body, thereby preventing the living body from being left behind. In a case where presentation that biological information has been detected is not carried out despite the existence of the living body in the vehicle, the occupant can find the failure of the biosensor.

[Aspect 2]

In the biological information presentation device of the present invention, the controller may start presentation via the presenter when the controller has acquired information indicating an occupant's intention to get on and the biosensor has detected biological information.

[Aspect 3]

In the biological information presentation device of the present invention, the information indicating the occupant's intention to get on may include information indicating at least any of activation of the travel power supply of the vehicle, activation of the auxiliary power supply of the vehicle, activation of the travel drive source of the vehicle, seating of the occupant, opening/closing of the door of the vehicle, unlock of the door of the vehicle, fastening of the seat belt of the vehicle, and operation of the operator of the vehicle.

According to the configuration described above, the detection processing of the living body by the biosensor can be started at a good timing.

[Aspect 4]

In the biological information presentation device of the present invention, the controller may continue presentation via the presenter at all times while the controller is acquiring information indicating the occupant's intention to get on and the biosensor is detecting biological information.

According to the configuration described above, the occupant can always recognize that the living body exists in the vehicle, and it is thus possible to prevent the living body from being left behind.

[Aspect 5]

In the biological information presentation device of the present invention, the controller may repeat the presentation via the presenter intermittently while the controller is acquiring the information indicating the occupant's intention to get on and the biosensor is detecting biological information.

According to the configuration described above, the occupant can intermittently recognize that the living body exists in the vehicle, leading to prevention of the living body from being left behind, and the power consumption by the biosensor can be suppressed low.

[Aspect 6]

In the biological information presentation device of the present invention, the presentation content presented via the presenter may further include the biological information of the occupant.

According to the configuration described above, the occupant can find that the living body exists in the vehicle, can also find the state of the living body, and can quickly respond to an abnormality in the living body, if any.

[Aspect 7]

In the biological information presentation device of the present invention, during the presentation operation via the presenter, if the controller acquires information indicating the occupant's intention to get out, the controller may change the mode of presentation via the presenter.

[Aspect 8]

In the biological information presentation device of the present invention, the information indicating the occupant's intention to get out may include information indicating at least any of stopping of the travel power supply of the vehicle, stopping of the auxiliary power supply of the vehicle, stopping of the travel drive source of the vehicle, leaving the seat of the occupant, opening/closing of the door of the vehicle, removal of the seat belt of the vehicle, and shift of the shift lever of the vehicle to the parking position.

According to the configuration described above, since the biosensor is always operated while the occupant exists in the vehicle, warning against leaving someone behind can be smoothly issued if the occupant indicates an intention to get out, and warning against leaving someone behind can be reliably set off to the occupant in a scene where there is a high possibility of leaving someone behind.

[Aspect 9]

In the biological information presentation device of the present invention, the change of mode of presentation via the presenter may include any of change in color of display on a display that displays the presentation content presented via the presenter, change in size of display on the display, change in design of display on the display, and change in output of warning sound.

According to the configuration described above, in a scene where there is a high possibility of leaving someone behind, it is possible to present warning against leaving someone behind in a presentation mode that strongly affects recognition of the occupant, and it is possible to reliably prevent leaving someone behind.

[Aspect 10]

In the biological information presentation device of the present invention, the controller may stop the presentation via the presenter if the biosensor becomes in a state of not detecting biological information, or if the presentation setting is canceled by an operation of the occupant.

According to the configuration described above, presentation can be stopped when presentation by the presenter is not necessary.

[Aspect 11]

The biological information presentation method of the present invention includes a step of detecting, by a biosensor, biological information of an occupant existing in a vehicle, and a control step of presenting, via a presenter, the fact that biological information has been detected if the biosensor detects the biological information.

According to the configuration described above, it is possible to achieve the same effects as those of the biological information presentation device according to the aspect 1 of the present invention.

[Aspect 12]

The biological information presentation program according to the present invention is a biological information presentation program for causing a computer to function as the biological information presentation device according to any of the above-described aspects 1 to 10, and is a program for causing a computer to function as the controller.

According to the configuration described above, it is possible to achieve the same effects as those of the biological information presentation device according to the aspect 1 of the present invention.

[Additional Notes]

The present invention is not limited to the embodiments described above, and various modifications can be made within the scope of the claims, and the embodiments obtained by appropriately combining the technical means disclosed in the different embodiments are also within the scope of the present invention.

While the invention has been described with reference to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

1. A biological information presentation device, comprising:
a biosensor configured to detect biological information of an occupant existing in a vehicle; and
a controller configured to present, via a presenter, a fact that biological information has been detected if the biosensor detects biological information.

2. The biological information presentation device according to claim 1, wherein
the controller starts presentation via the presenter when the controller has acquired information indicating an occupant's intention to get on and the biosensor has detected biological information.

3. The biological information presentation device according to claim 2, wherein
information indicating an occupant's intention to get on includes information indicating at least any of activation of a travel power supply of a vehicle, activation of an auxiliary power supply of a vehicle, activation of a travel drive source of a vehicle, seating of an occupant, opening/closing of a door of a vehicle, unlock of a door of a vehicle, fastening of a seat belt of a vehicle, and operation of an operator of a vehicle.

4. The biological information presentation device according to claim 1, wherein
the controller continues presentation via the presenter at all times while the controller is acquiring information indicating an occupant's intention to get on and the biosensor is detecting biological information.

5. The biological information presentation device according to claim 1, wherein
the controller repeats presentation via the presenter intermittently while the controller is acquiring information indicating an occupant's intention to get on and the biosensor is detecting biological information.

6. The biological information presentation device according to claim 1, wherein
a presentation content presented via the presenter further includes biological information of an occupant.

7. The biological information presentation device according to claim 1, wherein
during a presentation operation via the presenter, if the controller acquires information indicating an occupant's intention to get out, the controller changes a mode of presentation via the presenter.

8. The biological information presentation device according to claim 7, wherein
information indicating an occupant's intention to get out includes information indicating at least any of stopping of a travel power supply of a vehicle, stopping of an auxiliary power supply of a vehicle, stopping of a travel drive source of a vehicle, leaving a seat of an occupant, opening/closing of a door of a vehicle, removal of a seat belt of a vehicle, and shift of a shift lever of a vehicle to a parking position.

9. The biological information presentation device according to claim 7, wherein change of mode of presentation via the presenter includes any of change in color of display on a display that displays a presentation content presented via the presenter, change in size of display on the display, change in design of display on the display, and change in output of warning sound.

10. The biological information presentation device according to claim 1, wherein
the controller stops presentation via the presenter if the biosensor becomes in a state of not detecting biological information, or if presentation setting is canceled by an operation of an occupant.

11. A biological information presentation method, comprising:
a step of detecting, by a biosensor, biological information of an occupant existing in a vehicle; and
a control step of presenting, via a presenter, a fact that biological information has been detected if the biosensor detects biological information.

12. A non-transitory computer-readable medium storing biological information presentation program that, when executed by a processor, to cause a computer to function as the biological information presentation device according to claim 1, the biological information presentation program for causing a computer to function as the controller.
